(19) United States
(12) Patent Application Publication
(10) Pub. No.: US 2014/0066789 A1
(43) Pub. Date: Mar. 6, 2014

(54) SENSOR GUIDEWIRE

(71) Applicant: ASAHI INTECC CO. LTD., Nagoya-shi (JP)

(72) Inventors: Makoto NISHIGISHI, Owariasahi-shi (JP); Takayuki UTANI, Owariasahi-shi (JP)

(21) Appl. No.: 13/930,905

(22) Filed: Jun. 28, 2013

(30) Foreign Application Priority Data
Aug. 31, 2012 (JP) 2012-190945

(51) Int. Cl. A61B 5/0215

(52) U.S. Cl. CPC A61B 5/0215
USPC 600/486

(57) ABSTRACT

A sensor guidewire includes a sensor and a tubular body that covers the sensor. The tubular body includes a proximal blocking wall that is formed on a proximal side of a measurement portion of the sensor, a distal blocking wall that is formed on a distal side of the measurement portion of the sensor, and a hole that extends through the tubular body and through which blood flows into or flows out of the tubular body past the measurement portion of the sensor. The sensor is disposed on a proximal side of the hole. The proximal blocking wall and the distal blocking wall form a measurement chamber in the tubular body, and the sensor does not impede blood flow through the hole.

### CROSS REFERENCE TO RELATED APPLICATION

The present application claims priority to Japanese Patent Application No. 2012-190945 filed in the Japan Patent Office on Aug. 31, 2012, the entire contents of which are incorporated herein by reference.

### BACKGROUND

1. Field

The disclosed embodiments relate to a medical device. Specifically, the disclosed embodiments relate to a sensor guidewire.

2. Description of Related Art

To date, various guidewires have been proposed for guiding a catheter or the like that is inserted into a tubular organ or a body tissue, such as a blood vessel, an alimentary canal, or a ureter, and that is used for treatment and diagnosis.

For example, Japanese Unexamined Patent Application Publication No. 2003-265617 describes a sensor guidewire including a pressure sensor that is housed in a cylindrical metal casing that is disposed in a distal end portion of the sensor guidewire. Blood or the like flows into the sensor through a hole formed in the metal casing, and, for example, blood pressure is measured. The sensor includes a pressure sensitive element that is disposed at a position facing the hole.

Japanese Unexamined Patent Application Publication No. 2007-296354 describes a sensor guidewire including a sensor that is fitted into a recess formed in a core shaft.

### SUMMARY

The sensor guidewire described in Japanese Unexamined Patent Application Publication No. 2003-265617 has a problem in that, because the measurement portion of the sensor (pressure sensitive element) is disposed at a position facing the hole, blood flow through the hole is impeded by the sensor and the accuracy of measurement is low.

The sensor guidewire disclosed in Japanese Unexamined Patent Application Publication No. 2007-296354 has a problem in that it is difficult to perform measurement in a portion of a blood vessel where the amount of blood flow is small, such as a stenosis or an obstruction, because the measurement portion of the sensor might not be immersed in blood in such a portion of a blood vessel.

Accordingly, it is an object of the present invention to provide a sensor guidewire with which measurement using a sensor can be easily performed with high accuracy.

According to one embodiment, a sensor guidewire includes a sensor and a tubular body that covers the sensor. The tubular body includes a proximal blocking wall that is formed on a proximal side of a measurement portion of the sensor, a distal blocking wall that is formed on a distal side of the measurement portion of the sensor, and a hole that extends through the tubular body and through which blood flows into or flows out of the tubular body past the measurement portion of the sensor. The sensor is disposed on a proximal side of the hole.

With the sensor guidewire, the proximal blocking wall and the distal blocking wall form a measurement chamber in the tubular body, so that the measurement portion of the sensor can be immersed in blood without fail. Moreover, the sensor does not impede blood flow past the sensor. Therefore, measurement using the sensor can be easily performed with high accuracy.

### BRIEF DESCRIPTION OF THE DRAWINGS

FIG. 1 is a partial sectional view of a sensor guidewire according to a first embodiment.

FIG. 2 is a partial enlarged sectional view of the sensor guidewire taken along a plane different from that of FIG. 1.

FIG. 3 is a partial enlarged sectional view of a sensor guidewire according to a second embodiment.

FIG. 4 is a partial enlarged sectional view of a sensor guidewire according to a third embodiment.

FIG. 5 is a partial enlarged sectional view of a sensor guidewire according to a fourth embodiment.

### DETAILED DESCRIPTION

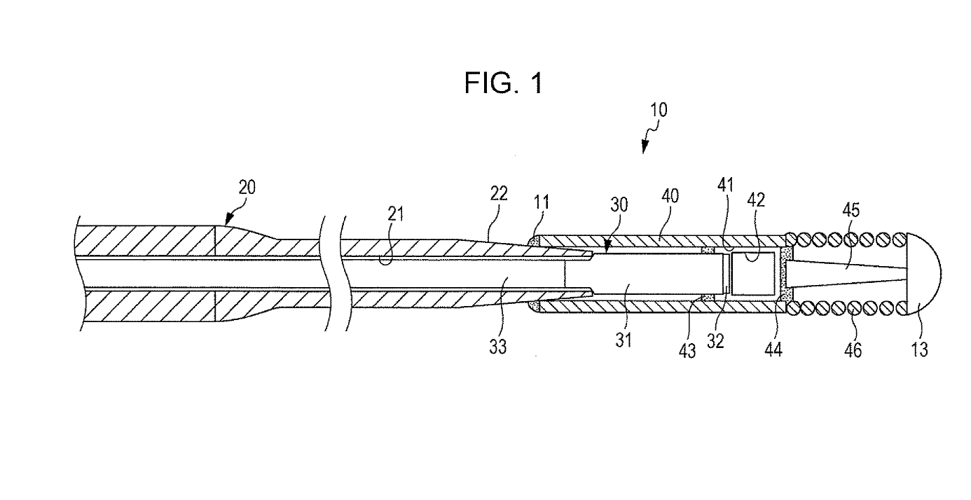

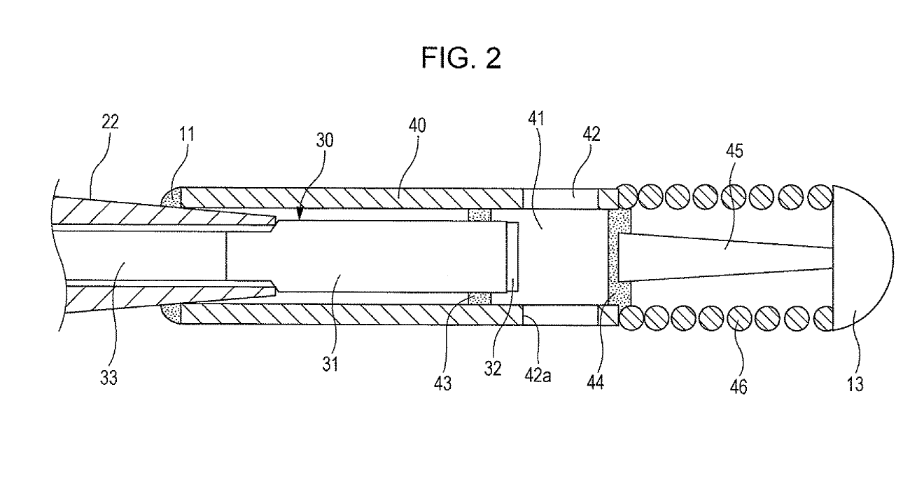

Referring to FIGS. 1 and 2, a sensor guidewire according to a first embodiment will be described. In FIGS. 1 and 2, the right side is the distal side, on which a distal end of the sensor guidewire, which is inserted into a human body, is located; and the left side is the proximal side, on which a proximal end (not shown) of the sensor guidewire, which is operated by an operator such as a doctor, is located. For ease of understanding the shapes of holes in a hypotube, FIG. 2 illustrates a sectional view of the guidewire that is taken along a plane passing through the holes.

A sensor guidewire 10 illustrated in FIG. 1 is used to treat a blood vessel of a heart or the like. The length of the sensor guidewire 10 is, for example, about 1900 mm. The sensor guidewire 10 includes a core shaft 20, a sensor 30 that is attached to the core shaft 20, and a hypotube 40 that covers the sensor 30.

The core shaft 20 has a cylindrical shape. The core shaft 20 has a hollow portion 21 formed therein and a taper portion 22 formed at a distal end thereof. The hollow portion 21 extends from the distal end to the proximal end of the core shaft 20 (not shown). The outside diameter of the taper portion 22 decreases toward the distal end. The material of the core shaft 20 is not particularly limited. In the present embodiment, the core shaft 20 is made of a stainless steel (SUS). Alternatively, the core shaft 20 may be made of a superelastic alloy such as a Ni—Ti alloy.

The sensor 30 includes a sensor body 31, a measurement portion 32 that is disposed at a distal end of the sensor body 31, and an optical fiber 33 extending from the sensor body 31 toward the proximal end. The sensor body 31 is attached to the distal end of the core shaft 20. The position of the measurement portion 32, which is located in a distal portion of the hypotube 40, will be described below in detail. The optical fiber 33 extends from the sensor body 31 through the hollow portion 21 of the core shaft 20 and is connected to an external apparatus (not shown). Information detected by the sensor 30 of the sensor guidewire 10 is used for various operations and diagnoses. In the present embodiment, the sensor 30 measures the blood pressure in a blood vessel. Alternatively, a sensor for measuring other information may be used.

A proximal end of the hypotube 40 is brazed to an outer surface of the taper portion 22 of the core shaft 20 through a proximal brazed portion 11. As illustrated in FIG. 2, the hypotube 40 includes a proximal blocking wall 43, a distal blocking wall 44, and a pair of holes 42. The proximal blocking wall 43 is formed on the proximal side of the measurement portion 32 of the sensor 30. The distal blocking wall 44 is formed on the distal side of the measurement portion 32 of the sensor 30. The holes 42 extend through the hypotube 40, and blood flows into or flows out of the measurement portion 32 of the sensor 30 through the holes 42. The proximal blocking wall 43 and the distal blocking wall 44 form a measurement chamber 41 in the hypotube 40. The measurement portion 32 of the sensor 30 is disposed in the measurement chamber 41. The holes 42 are formed in portions of the hypotube 40 that are located opposite each other in the radial direction. The holes 42 have square shapes, and the peripheral surfaces of the holes 42 extend in the radial direction of the hypotube 40. The sensor 30 is disposed on the proximal side of the holes 42. The measurement portion 32 of the sensor 30 is not located along a path connecting the holes 42 to each other, but is located at a position immediately on the proximal side of the path. Thus, the sensor 30 does not impede blood flow between the holes 42. Moreover, the measurement portion 32 of the sensor 30 can detect blood flow without fail at a position immediately on the proximal side of the blood flow. In particular, the sensor 30 has high sensitivity in a case where the position of the distal end of the measurement portion 32 in the axial direction of the hypotube 40 is the same as that of a peripheral surface 42a, which is a proximal portion of the peripheral surface of each of the holes 42.

In the present embodiment, the hypotube 40 is made of a metal such as a stainless steel (SUS). In the present embodiment, the proximal blocking wall 43 and the distal blocking wall 44 are made of a brazing alloy. That is, the proximal blocking wall 43 and the distal blocking wall 44 are made of a material that has fluidity when the blocking walls 43 and 44 are formed and that solidifies after the blocking walls 43 and 44 are formed. Examples of the brazing alloy include an aluminum alloy solder, silver solder, gold solder, zinc, a Sn—Pb alloy, a Sn—Au alloy, a Pb—Ag alloy, a Sn—Ag alloy, a Au—Sn alloy, and a Au—Si alloy.

A brazing alloy that forms the proximal blocking wall 43 joins a portion of the sensor 30 that is immediately on the proximal side of the measurement portion 32 to an inner wall of the hypotube 40 without forming a gap therebetween. Thus, the proximal blocking wall 43 supports the measurement portion 32 of the sensor 30. A brazing alloy that forms the distal blocking wall 44 joins the distal end of the hypotube 40, the proximal end of a distal end shaft 45, and the proximal end of a coil 46 to each other without forming a gap therebetween. The distal end shaft 45 is tapered so that the outside diameter thereof decreases toward the distal end. The coil 46 covers the entirety of the distal end shaft 45. A tip portion of the distal end shaft 45 and a tip portion of the coil 46 are brazed to each other through a distal tip 13. The distal tip 13 has a hemispherical surface on a distal side thereof.

As described above, in the sensor guidewire 10, the hypotube 40 includes the proximal blocking wall 43, which is formed on the proximal side of the measurement portion 32 of the sensor 30; the distal blocking wall 44, which is formed on the distal side of the measurement portion 32 of the sensor 30; and the holes 42, which extend through the hypotube 40 and through which blood flows into or flows out of the measurement portion 32 of the sensor 30. Moreover, the sensor 30 is disposed on the proximal side of the holes 42. Therefore, the proximal blocking wall 43 and the distal blocking wall 44 form the measurement chamber 41 in the hypotube 40, so that the measurement portion 32 of the sensor 30 is immersed in blood without fail. Moreover, the sensor 30 does not impede blood flow through the holes 42. As a result, with the sensor guidewire 10, measurement using the sensor 30 can be easily performed with high accuracy.

In the sensor guidewire 10, the proximal blocking wall 43 supports the measurement portion 32 of the sensor 30. Therefore, it is not necessary to provide the sensor guidewire 10 with an independent member for fixing the sensor 30 in place, so that the structure of the sensor guidewire 10 can be simplified.

In the sensor guidewire 10, the proximal blocking wall 43 and the distal blocking wall 44 are made of a material that has fluidity when the blocking walls 43 and 44 are formed and that solidifies after the blocking walls 43 and 44 are formed. Therefore, it is easy to form the measurement chamber 41 and it is possible to provide the measurement chamber 41 with high hermeticity.

In the sensor guidewire 10, the hypotube 40 is made of a metal, and the proximal blocking wall 43 and the distal blocking wall 44 are made of a brazing alloy. Therefore, it is easy to form the measurement chamber 41 having particularly high hermeticity in the metal hypotube.

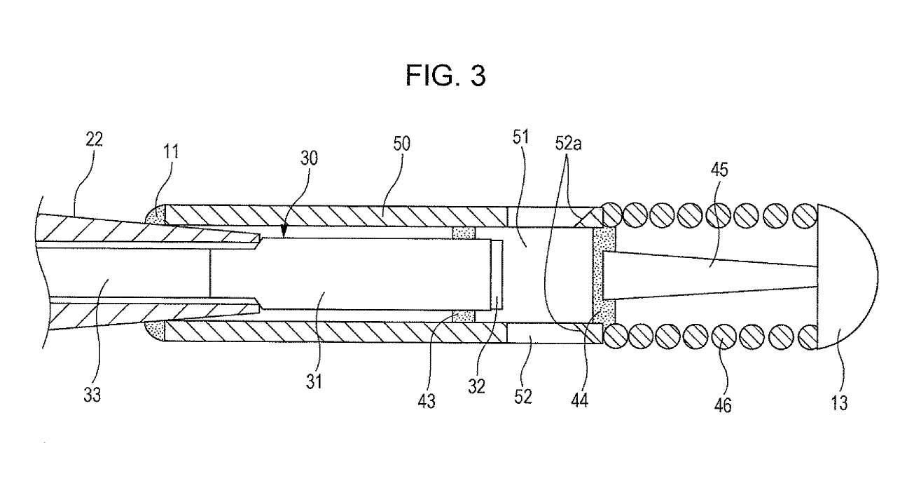

Referring to FIG. 3, a sensor guidewire according to a second embodiment of the present invention will be described. In FIG. 3, the right side is the distal side and the left side is the proximal side, as in FIG. 1. The components of the sensor guidewire the same as those of the first embodiment will be denoted by the same numerals and description of such components will be omitted. The following description will focus on the differences from the first embodiment.

The sensor guidewire according to the second embodiment differs from that of the first embodiment in the shapes of the holes in the hypotube. As illustrated in FIG. 3, in a hypotube 50 according to the second embodiment, a distal portion of the peripheral surface of each hole 52 is an inclined surface 52a along which the inside dimension of the hole 52 increases from the inside toward the outside of the hypotube 50.

In the second embodiment, the inside dimension of each hole 52 is increased by forming the inclined surface 52a. Therefore, in addition to the effect obtained by the first embodiment, the sensor guidewire according to the second embodiment has an effect that blood flow can be more efficiently guided to a measurement chamber 51 even when the sensor guidewire is inserted into a blood vessel in which the amount of blood flow is small.

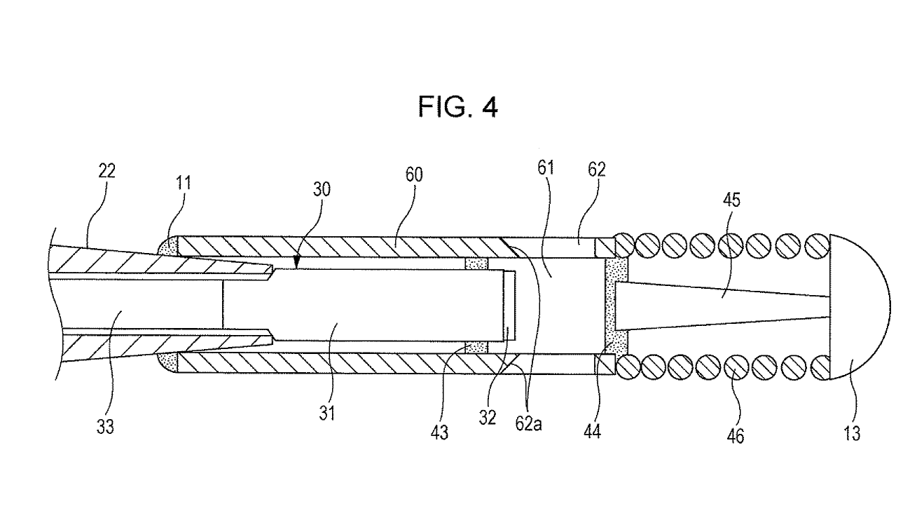

Referring to FIG. 4, a sensor guidewire according to a third embodiment of the present invention will be described. In FIG. 4, the right side is the distal side and the left side is the proximal side, as in FIG. 1. The components the same as those of the first and second embodiments will be denoted by the same numerals and description of such components will be omitted. The following description will focus on the differences from the first and second embodiments.

The sensor guidewire according to the third embodiment differs from those of the first and second embodiments in the shapes of the holes in the hypotube. As illustrated in FIG. 4, in a hypotube 60 according to the third embodiment, a proximal portion of the peripheral surface of each hole 62 is an inclined surface 62a along which the inside dimension of the hole 62 increases from the inside toward the outside of the hypotube 60. The inside dimension of each hole 62 is increased on the proximal side with consideration of the fact that blood flows in a blood vessel from the proximal side toward the distal side.

In the third embodiment, the inside dimension of the hole 62 is increased on the proximal side, from which blood flows in a blood vessel, by forming the inclined surface 62a. Therefore, in addition to the effect obtained by the first embodiment, the sensor guidewire has an effect that blood flow can be more efficiently guided to a measurement chamber 61 even when the sensor guidewire is inserted into a blood vessel in which the amount of blood flow is small.

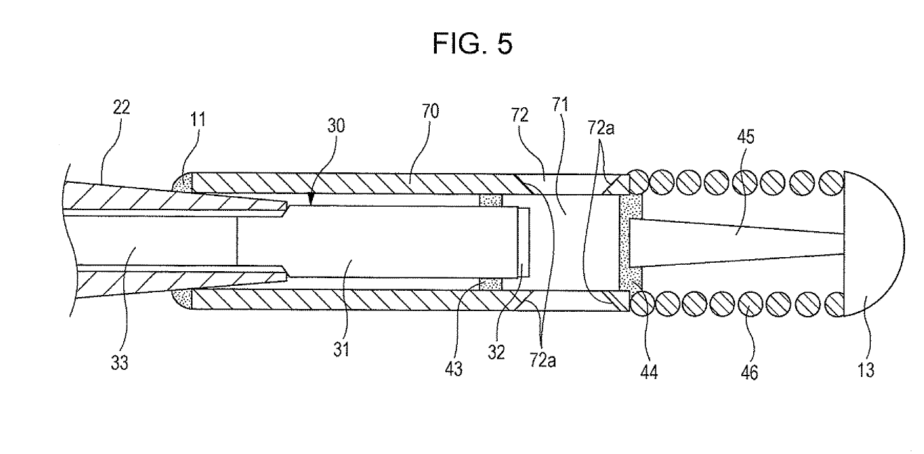

Referring to FIG. 5, a sensor guidewire according to a fourth embodiment of the present invention will be described. In FIG. 5, the right side is the distal side and the left side is the proximal side, as in FIG. 1. The components the same as those of the first to third embodiments will be denoted by the same numerals and description of such components will be omitted. The following description will focus on the differences from the first to third embodiments.

The sensor guidewire according to the fourth embodiment differs from those of the first to third embodiments in the shapes of the holes in the hypotube. As illustrated in FIG. 5, in a hypotube 70 according to the fourth embodiment, the entirety of the peripheral surface of each hole 72 is an inclined surface 72a along which the inside dimension of the hole 72 increases from the inside toward the outside of the hypotube 70.

In the fourth embodiment, the entirety of the peripheral surface of each hole 72 is the inclined surface 72a, along which the inside dimension of the hole 72 increases from inside toward outside. Therefore, the sensor guidewire according to the fourth embodiment has an effect that blood can flow into a measurement chamber 71 more efficiently than into a measurement chamber according to any of the first to third embodiments.

The embodiments described above are only examples and do not limit the present invention. The disclosed embodiments can be modified in various ways without departing from the invention.

For example, in the embodiments described above, the hypotube 40 is made of a metal, and the proximal blocking wall 43 and the distal blocking wall 44 are made of a brazing alloy. Alternatively, the hypotube may be made of a resin, and the proximal blocking wall and the distal blocking wall may be made of a resin adhesive. In this case, it is possible to form a measurement chamber having a particularly high hermeticity in the resin hypotube.

What is claimed is:

1. A sensor guidewire comprising:
a sensor; and
a tubular body that covers the sensor,
wherein the tubular body includes
a proximal blocking wall that is formed on a proximal side of a measurement portion of the sensor,
a distal blocking wall that is formed on a distal side of the measurement portion of the sensor, and
a hole that extends through the tubular body and through which blood flows into or flows out of the tubular body past the measurement portion of the sensor, and
wherein the sensor is disposed on a proximal side of the hole.

2. The sensor guidewire according to claim 1,
wherein at least a part of a peripheral surface of the hole is an inclined surface along which an inside dimension of the hole increases from an inside toward an outside of the tubular body.

3. The sensor guidewire according to claim 2,
wherein the inclined surface is formed at least in a proximal portion of the peripheral surface.

4. The sensor guidewire according to claim 1,
wherein the proximal blocking wall supports the measurement portion of the sensor within the tubular body.

5. The sensor guidewire according to claim 1,
wherein the proximal blocking wall and the distal blocking wall are made of a material that has fluidity when the blocking walls are formed and that solidifies after the blocking walls are formed.

6. The sensor guidewire according to claim 5, wherein
the tubular body is made of a metal, and
the proximal blocking wall and the distal blocking wall are made of a brazing alloy.

7. The sensor guidewire according to claim 5, wherein
the tubular body is made of a resin, and
the proximal blocking wall and the distal blocking wall are made of a resin adhesive.

8. The sensor guidewire according to claim 1, wherein the hole has a square shape.

9. The sensor guidewire according to claim 1, wherein the tubular body, the proximal blocking wall, and the distal blocking wall define a measurement chamber.

10. The sensor guidewire according to claim 9, wherein the hole is disposed in portions of the tubular member that are opposite in a radial direction defining a path from a first opening to a second opening through the measurement chamber.

11. The sensor guidewire according to claim 10, wherein the measurement portion of the sensor is disposed on a proximal side of the path.

12. The sensor guidewire according to claim 1, wherein a distal end of the measurement portion of the sensor and a peripheral surface of the hole are disposed at substantially a same axial position.

13. The sensor guidewire according to claim 2,
wherein the inclined surface is formed at least in a distal portion of the peripheral surface.

14. The sensor guidewire according to claim 2,
wherein the inclined surface is formed along an entirety of the peripheral surface.

15. The sensor guidewire according to claim 4, wherein the proximal blocking wall directly joins the sensor to an inner wall of the tubular member without forming a gap therebetween.

16. The sensor guidewire according to claim 1, wherein the distal blocking wall joins a distal end of the tubular member, a proximal end of a distal end shaft, and a proximal end of a coil without forming a gap therebetween.

17. The sensor guidewire according to claim 1, wherein the measurement portion of the sensor does not obstruct the hole.

18. The sensor guidewire according to claim 1, wherein the hole defines a passage that extends through the tubular member, and the measurement portion of the sensor does not extend into the passage.
